(19) United States
(12) Patent Application Publication
(10) Pub. No.: US 2018/0021162 A1
(43) Pub. Date: Jan. 25, 2018

(54) PROTECTIVE LIMB BRACE

(71) Applicants: Scott BLACKBURN, South Glenelg (AU); Charles BRUNNTHALER, South Glenelg (AU)

(72) Inventors: Scott BLACKBURN, South Glenelg (AU); Charles BRUNNTHALER, South Glenelg (AU)

(21) Appl. No.: 15/548,644

(22) PCT Filed: Feb. 3, 2016
(86) PCT No.: PCT/AU2016/000024

§ 371 Date: Aug. 3, 2017

(30) Foreign Application Priority Data
Feb. 3, 2015 (AU) 2015900316

(51) Int. Cl. A61F 5/058

(52) U.S. Cl. CPC A61F 5/05825; A61F 5/0585; A61F 5/05858

(57) ABSTRACT

A portable splint assembly for fixation to a limb. The splint assembly comprises at least one elongated member having first and second fee ends; and at least one fastener which enables fastening of the splint to a limb. The at least one elongated member has a long axis along its longitudinal extent extending between the free ends and at least one transverse shorter axis. The elongated member is resiliently deformable to allow folding and/or or rolling about at least one said axes. The least one elongated member moves between a first state in which the elongated member is completely folded and/or rolled up and a second state in which the at least one elongated member is unfolded and/or unrolled to enable working engagement with a limb.

### BACKGROUND

The present invention relates to first aid devices and assemblies and more particularly relates to such devices used in rescue and emergency events requiring immobilisation of damaged limbs. The present invention further relates to medical appliances and more particularly relates to a limb brace for use in supporting limbs which have been injured or which require rehabilitation. The present invention also relates to limb splints used in first aid management of broken bones in remote locations and accident environments. The invention more specifically relates to a limb brace which can be rolled and/or folded up and which has improved versatility, portability, durability and ease of use. The invention further relates to a portable limb splint which temporarily immobilises damaged limbs as initial first aid and which can be applied with minimal discomfort to a victim irrespective of the size of the limb or patient. The invention further provides a splint which includes flexible release straps which allows efficient setting and removal from a limb. The limb splint according to the invention is adjustable to accommodate a variety of leg or arm positions so as to orient the leg or arm in a disposition best conducive to treatment. The invention further provides a limb support assembly allowing multiple degrees of freedom for placing a limb such as a but not limited to leg or arm at selective degrees of restraint.

### PRIOR ART

A splint is a common device used in the treatment of human injuries such as damaged joints and bones. The purpose of the splint is to immobilise and support the injured area of the body to prevent movement and further injury. Splints commonly consist of a flat or rigid surface which is applied to the injured area and attached by stirrups, bandages or other securement means to stabilise the area. Splints are often applied by first responder emergency personnel to immobilise a fractured limb, but are also commonly used by lay persons for the same purpose until medical treatment can be sought. Makeshift splits must often be created when an injury occurs, as it is not common for splints to be contained in standard first aid kits as they are bulky and not easily transportable. Many splits are also specifically designed far various limbs, for example, legs or arms and are not adaptable for use on other parts of the body. To carry various types of splints in a first aid kit to suit different limbs, takes up space, is cumbersome and impractical.

There are in existence various limb splint devices intended to immobilise a limb. Splints are used in first aid kits a ad also by rescuers tor immobilisation of limbs when persons have been injured. A splint is a device which is intended to secure a broken limb to inhibit pain due to mobilisation and to keep the limb straight or in its post trauma position to minimise the risk of further damage during transit to a treatment centre. Conventional splints are often rigid devices with little or no inherent flexibility. There are known splints which are moulded from plastics and which provide a cradle for limb support.

Leg injuries are common in a variety of sports and often require support either immediately following the injury or post operatively. In the event of a sporting injury, the leg may require support to relieve pain or to prevent further injury. Currently there exist various splints and the like to hold the limb securely until the patient can receive orthopaedic treatment.

By way of another example of the prior art, U.S. Pat. No. 6,953,442 discloses a knee brace having first and second arms constituting a pair of left and right arms fixed to the bilateral thigh sides along the user's femur. The brace has third and fourth arms constituting a pair of left and right arms fixed to the shank bilateral sides along the user's tibia. The lower ends of the first and second arms and the corresponding upper ends of the third and fourth arms are pivotally connected together to provide two pairs of upper and lower arms, so that the third and fourth arms are allowed to pivotally move with a pivotal part as the center correspondingly to the knee motion. Gears and guide grooves which respectively have racks meshing with the gears, extend in parallel to a femur or tibia axial center and provided opposite to the ends of the arms constituting the pairs of left and right arms. The aforesaid brace is the type used for rehabilitation of serious leg injuries such as after surgery. It is a complicated device designed for a specific purpose and is not suitable for field use.

U.S. Pat. No. 5,060,640 discloses a knee brace for supporting an injured interior cruciate ligament by using straps that produce a forced couple on the knee joint to keep the tibia properly behind the femur while allowing for adjustment of the amount of anti-drawering to fit the individual needs of the patient. The knee brace may utilize a quadricentric hinge that closely approximates the natural movement of the knee joint and allows for adjustment of the knee brace to account for the natural curvature of the individual patient's leg bones.

In another example of the prior art, U.S. Pat. No. 5,419,754 discloses a knee brace comprising upper and lower leg-engaging/embracing parts (thigh and calf pieces) connectable around upper and lower parts of the leg above and below the knee joint with the upper and lower leg embracing parts having upper and lower brace members respectively interconnected by at least one joint, wherein the at least one joint means each comprises at least one link member pivotally connected at pivot points to rotate about a pivotal axis on each of the brace members with the at least one link member being provided for holding brace members together, and wherein the end region of a first said brace member has a slot defined between two end arm portions and within the slot space for forming a defining portion thereof. This is a complicated arrangement for a specific purpose and is not suitable for convenient filed use for simply supporting a leg.

The known leg braces have been limited in their applications because they are restricted in their degrees of freedom and are each built for a specific limited purpose. Nor are they suitable for field use. One of the problems with the known leg splints is that they are difficult to set and maintain an optimum leg position and orientation. They take up space in rescue vehicles and are hard to fix to a damaged let.

There is a long felt want in the industry to provide an alternative to the known braces and leg support assembles and particularly those which are used in the field. Although the prior art teaches the use of leg braces there is a long felt want in the field to provide a leg splint which provides an alternative to the prior art limb braces and allows a leg to be retrained at selective tension to reduce pain and discomfort.

One object of this invention is to provide a foldable splint that alleviates the above problems, or at least provides the public with a useful alternative.

### INVENTION

The present seeks to ameliorate the aforesaid disadvantages of the prior art by providing a medical appliance and more particularly a leg splint assembly for use in supporting limbs which have been injured or which require medical care or treatment. More particularly the invention provides a leg brace assembly which is portable and adjustable to accommodate a variety of leg positions so as to orient a leg in a disposition best conducive to treatment. The invention further provides a leg support assembly allowing multiple degrees of freedom for placing a leg at selective elevations and attitudes.

It is one object of the invention to provide an improved limb splint which is simple to operate in the field and which has multiple degrees of freedom. It is a further object of the invention to provide an improved limb brace assembly which provides a simple alternative to the known braces and which may be disposed in multiple orientations. The present invention provides a useful alternative to the known splint devices. More particularly the present invention provides a self contained splint which is easy to store and operate in the field with minimal disruption to the patient. There has thus been outlined, rather broadly, the more important features of the invention in order that the detailed description thereof that follows may be better understood, and in order that the present contribution to the art may be better appreciated.

There are, of course, additional features of the invention that will be described hereinafter and which will form the subject matter of the claims. In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be utilized as a basis tor the designing of other structures, methods and systems for carrying out the several purposes of the present invention. It is important, therefore, that the claims be regarded as including such equivalent constructions insofar as they do not depart from the spirit and scope of the present invention.

The present invention allows rescuers, paramedics and first aid providers to conveniently apply a splint to a damaged limb with minimal discomfort to a patient. The splint is particularly useful in remote locations as it is lightweight, easy to fit and takes up minimal space in first aid inventories. The splint also allows any person including paramedics and the like to readily fit the splint virtually without movement of the damaged limb. This is effected by feeding straps conveniently under the limb. This avoids unnecessary pain and is particularly useful remote areas as it can be folded and stored in kit bags, first aid containers and in back packs. The capacity to fit the splint without movement of the limb avoids further damage a limbs.

The splint allows medics and rescuers to adopt safe practice earlier in a rescue. If farther damage to a limb is done while removing a patient from an accident site this can complicate future management of the injury. More injury can be prevented if splinting can be achieved with immobilisation avoiding injury occasioned by rough treatment of a patient during an actual rescue. This eliminates constant undesirable movement of the patient. In a first aspect the invention comprises a sheet a rectangular plastic where the traverse planes are longer than the longitudinal planes and wherein the traverse edges contain tabs and slots that when mated form a hall-cylindrical or other shape which can be used as a splint and wherein the plastic can also be folded for storage. It should be noted that any one of the aspects mentioned herein may include any of the features of any of the other aspects mentioned above and may include any of the features of any of the embodiments described below as appropriate.

In its broadest form the present invention comprises:

a splint assembly for fitting to a limb of a patient, the assembly comprising a splint body comprising at least one stiff elongated member to which are attached a plurality of flexible straps; wherein the straps are attached transversely relative to a longitudinal axis through the stiff elongated member, each strap having free ends which engage a free end of either same strap or another strap.

In another broad form, the present invention comprises: a splint assembly comprising a splint body capable of being folded between a folded state when the spirit is not in use and an unfolded state in which the splint envelops the limb of a patient; and at least one strap to secure the splint to a limb; the straps arranged to surround a limb of a patient and secure the splint to the limb thereby splinting the limb.

In another broad form, the present invention comprises:

a splint assembly comprising cooperating splint elements each having a plurality of flexible straps transversely disposed relative to a longitudinal extent of the strut member, the straps arranged to surround a limb of a patient and secure the strut to the limb thereby splinting the limb; wherein each said flexible straps are adjustable to selectively alter the tightness of the splint. two cooperating splint elements each comprising a stiff member and a plurality of flexible straps.

The present invention provides an alternative to the known prior art and the shortcomings identified. The foregoing and other objects and advantages will appear from the description to follow. In the description reference is made to the accompanying representations, which forms a part hereof, and in which is shown by way of illustration specific embodiments in which the invention may be practiced.

These embodiments will be described in sufficient detail to enable those skilled in the art to practice the invention, and it is to be understood that other embodiments may be utilised and that structural changes may be made without departing from the scope of the invention.

The following detailed description is, therefore, not to be taken in a limiting sense, and the scope of the present invention is best defined by the appended claims.

### BRIEF DESCRIPTION OF DRAWINGS

The present invention will now be described in more detail according to a preferred but non limiting embodiment and with reference to the accompanying drawings wherein:

FIG. 1 shows a plan view of an element of the splint assembly according to one embodiment;

FIG. 2 shows the element of FIG. 1 partially engaged with a like splint element prior to affixation to a limb.

FIG. 3 shows the assembly of FIG. 21 as it appears when secured to a limb.

FIG. 4 shows with like numbering the splint element of FIG. 1 previously described rolled up and prepared for storage.

FIG. 5 shows a folded view of a limb splint according to an alternative embodiment.

FIG. 6 shows the limb splint of FIG. 5 partially unfolded.

FIG. 7 shows the limb splint of FIG. 5 unfolded along its longitudinal extent.

FIG. 8 shows the limb splint of FIG. 7 partially unfolded across its transverse extent.

FIG. 9 shows the limb splint of FIG. 7 fully unfolded across its transverse extent.

FIG. 10 shows the limb splint enveloping a patient leg and secured with a strap.

FIG. 11 shows an exploded view of enlarged detail of a connection regime for connecting fastening straps to the splint.

FIG. 12 shows enlarged detail of the connection assembly of FIG. 11 assembled

FIG. 13 shows an abbreviated enlarged view of the splint and fastening strap extending through a slot and wound around the outside of the splint.

FIG. 14 shows a fully folded limb splint according to a further alternative embodiment.

FIG. 15 shows the limb splint of FIG. 14 partially unfurled and including fastening straps.

FIG. 16 shows the splint of FIG. 14 presented to an arm of a patient prior to engagement.

FIG. 17 shows the splint of FIG. 14 partially installed on a patient arm

FIG. 18 shows an enlarged view of the engagement between a fastening strap and abbreviated splint of FIG. 14.

FIG. 19 shows a perspective view of a segmented limb splint according to a further alternative embodiment.

FIG. 20 shows the limb splint of FIG. 19 with a segment isolated from the main splint.

FIG. 21 shows the segmented limb splint in a configuration suitable for use in splinting an arm.

### DETAILED DESCRIPTION

The following detailed description of the invention refers to the accompanying drawings. Wherever possible, the same reference numbers will be used throughout the drawings and the following description to refer to the same and like parts. Dimensions of certain parts shown in the drawings may have been modified and/or exaggerated for the purposes of clarity or illustration. The examples referred to herein are illustrative and are not to be regarded as limiting the scope of the invention. While various embodiments of the invention have been described herein, it will be appreciated that these are capable of modification, and therefore the disclosures herein are not to be construed as limiting of the precise details set forth, but to avail such changes and alterations as fall within the purview of the description. Although the method and apparatus aspects of the invention will be described with reference to their application to the embodiments described it will be appreciated that the invention can be embodied in alternative splints.

In a folded state the splint to be described herein is compact and easily transportable. For example it would fit easily into standard first aid kits or would be able to be kept in a convenient place in a user's home, workplace or car. This is an improvement on known splints which cannot be folded or compacted for the purposes of storage and transport. Preferably, the splint is made from a pre-formed plastics material of a suitable strength and flexibility. However it is to be understood that alternate materials could be used provided the splint is foldable about at least one axis (transverse and/or longitudinal). According to a preferred embodiment, the splint includes an elongated member which consists of two longitudinal folds and at least two traverse folds. When the splint is viewed in an unfolded state a longitudinal dimension is greater than a transverse dimension, and the longitudinal planes are greater than the traverse planes.

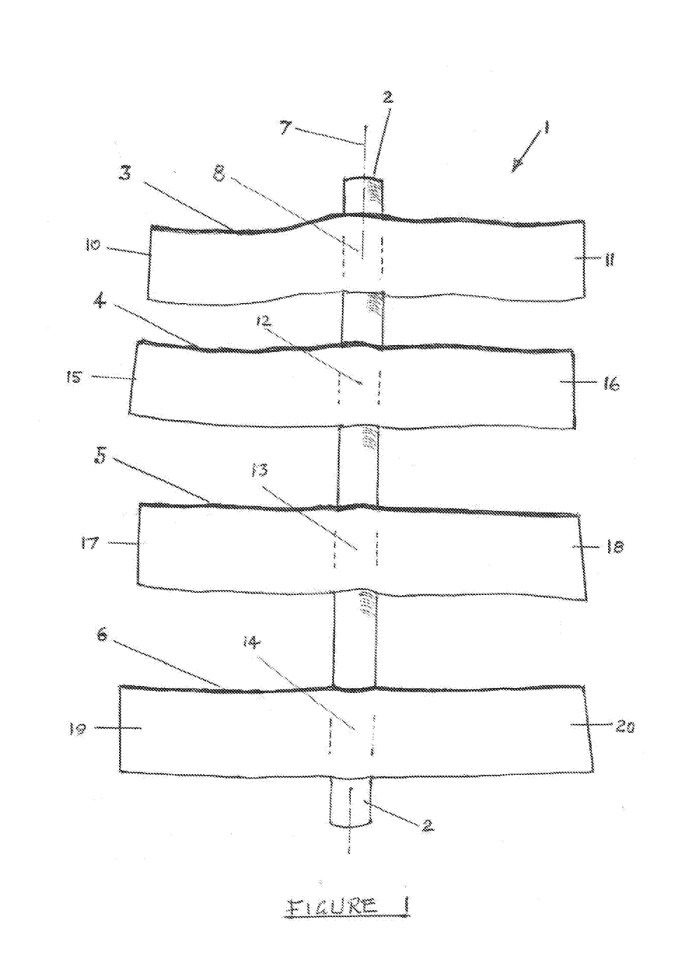

Referring to FIG. 1 there is shown a splint element 1 comprising a stiff strut 2 to which is attached a plurality of flexible straps 3, 4, 5 and 6. Each strap is fixed to stiff member 2 such that it is transverse to longitudinal axis 7. Strap 3 is preferably fitted midway along it length at connection point 8. Strut 2 is preferably manufactured from a lightweight but stiff plastics material.

It will be appreciated that other materials can be used such as timber preferably lightweight, composite plastics, recycled plastics, graphite (carbon fibre) and metals such as but not limited to aluminium. The material selected must be stiff enough to withstand forces applied during splinting including external impact on the splint assembly

Flexible strap 3 is preferably manufactured from a flexible soft material which is of sufficient strength to withstand hoop and other stresses when splint element 1 is applied in use. Strap 3 preferably comprises Velcro™ which allows adjustable length fastening to accommodate limb geometry and quick release and attachment. Strap 3 is attached transversely relative to longitudinal axis 7 through the stiff member via connection point 8 midway between free ends 10 and 11 of strap 3.

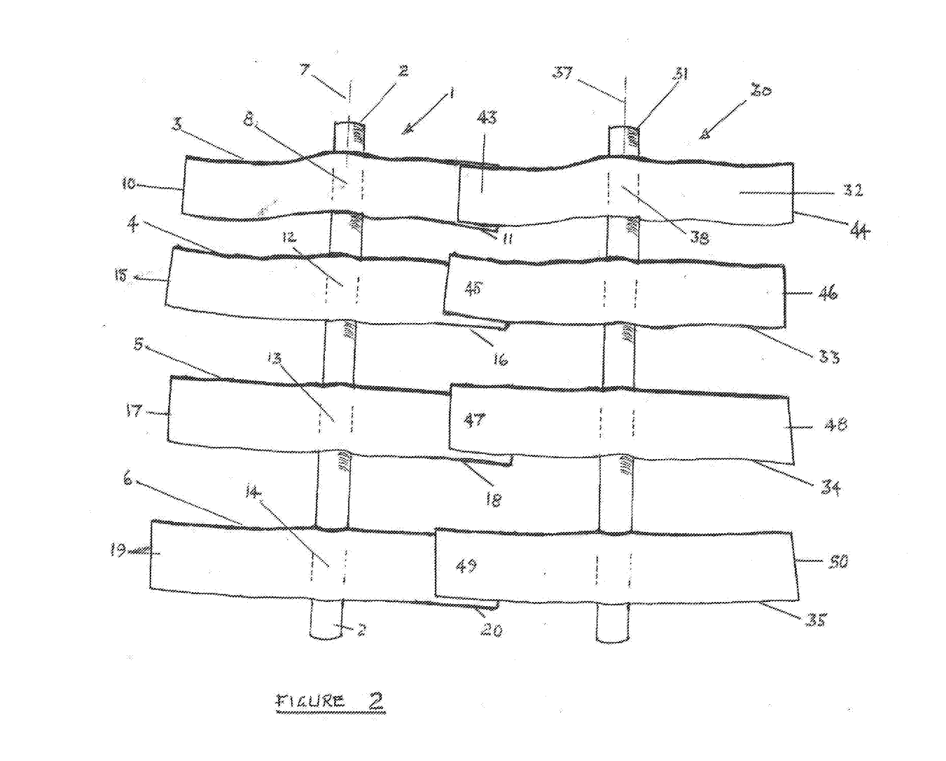

Likewise straps 4, 5 and 6 are connected to member 2 at respective connection points 12, 13 and 14. Strap 4 terminates at free ends 15 and 16. Strap 5 terminates in free ends 17 and 18 and strap 6 terminates in free ends 19 and 20. The free ends of each strap engage a free end of the same strap or of a strap associated with a second splint element (see FIG. 2 described below), either same strap or another strap. The splint element 1 of FIG. 1 can be used as a rudimentary splint for small children or for a small limb where one stiffener is sufficient. When element 1 is used alone free ends of each strap will mutually engage to form a loop which can be tightened as required. Thus ends 10 and 11 of strap 3 mutually engage. Likewise ends 15 and 16 of strap 4 engage, ends 17 and 18 of strap 5 engage and ends 19 and 20 of strap 6 engage.

FIG. 2 shows the element 1 of FIG. 1 partially engaged with a like splint element 30 prior to affixation to a limb. Element 30 comprises a stiff strut member 31 to which is attached a plurality of flexible straps 32, 33, 34 and 35. Each strap is fixed to stiff strut 31 such that it is transverse to longitudinal axis 37. Strap 32 is preferably fitted midway along it length at connection point 38 preferably midway between free ends 38 and 39 of strap 32. Member 31 is preferably manufactured from a lightweight but stiff plastics material. Similar materials are used for element 30 as were described for element 2.

Strap 32 as with all straps preferably comprises Velcro™ which allows adjustable length fastening to accommodate limb geometry and quick release and attachment. Strap 32 is attached transversely relative to longitudinal axis 37 through the strut 31 via connection point 38.

A variety of connections may be adopted such as studs, glue, slot engagement sewn loop. In one embodiment the strap can be adjustable along the length of the struts 2 and 31 and laterally relative to strut 2 and 31. Likewise straps 33, 34 and 35 are connected to strut 31 at respective connection points 40, 41 and 42. Strap 32 terminates at free ends 43 and 44. Strap 33 terminates in free ends 45 and 46, strap 34 terminates in free ends 47 and 48 and strap 35 terminates in free ends 49 and 50. The free ends of each strap engage a corresponding free end of a strap associated with a splint element 30.

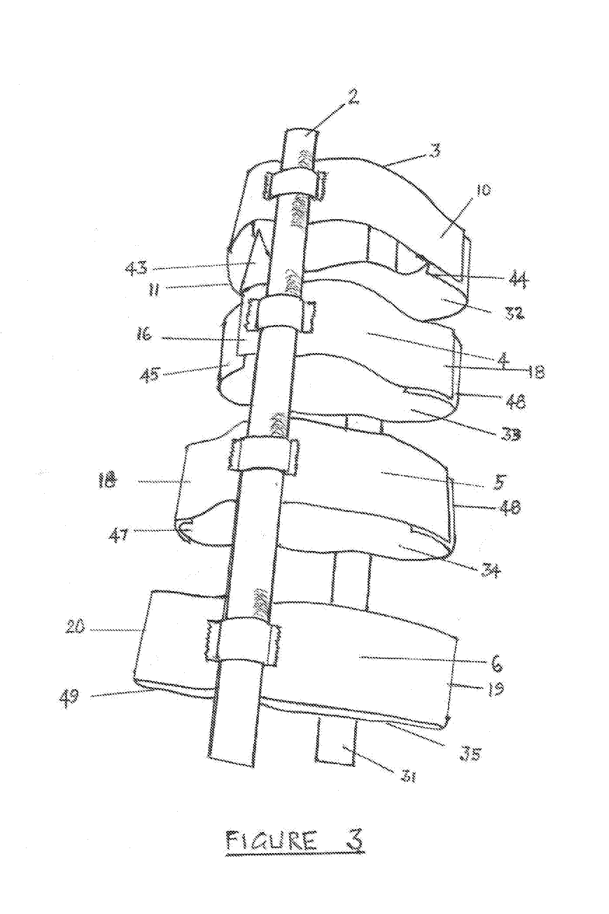

FIG. 2 shows the splint assembly as it would appear laid out before securing to a limb. FIG. 3 shows with corresponding numbering the assembly of FIG. 2 as it appears when secured to a limb. In the embodiment shown, four loops are formed which would in use, embrace a limb. A user can elect how tight to fit the splint by adjusting the extent of overlap between engaging ends of the opposing straps. Thus strap 3 engages strap 32 via ends 11 and 43 and ends 10 and 44. Straps 4 and 33 engage via ends 16 and 45 and 15 and 46. Straps 5 and 34 engage via ends 18 and 47 and 17 and 48. Straps 6 and 35 engage via ends 20 and 49 and 19 and 50.

The user can make incremental adjustments between the extent of overlap between any of the engaging ends to alter the splinting forces required. This is ideally performed using Velcro fastening but alternative methods of fastening may be adopted. The straps are adjusted to provide required tightness. This can provide more comfort to the patient but still allows best paramedical practice. The present invention therefore reduces unnecessary patient limb movements when a patient must be removed from an accident site without unwanted movement of the limb. Once a patient is splinted the patient can then be transferred to further transport i.e. ambulance, flying doctor, helicopter or another extraction vehicle.

Each of struts 2 and 31 are cut to a length suitable for a particular patient. For example, adult and child's versions can be made available. Alternatively the splint elements are provided in one or a small number of standard sizes which can be scissor or blade cut to length depending upon the nature of the patient. Alternatively excess length of a standard size can be folded over or rolled over to a required size (length) for a particular limb. Material selection for the struts 1 and 30 is important for satisfying the aforesaid objectives of achieving the optimal length from a standard size, rolling up for storage or cutting excess length. An analogy is a tape measure which is stiff enough for its purpose but can also roll up for storage.

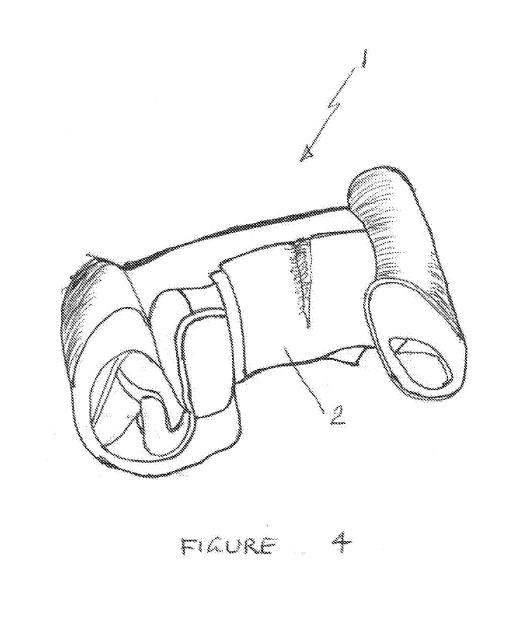

FIG. 4 shows with like numbering splint element 1 previously described rolled up and prepared for storage. Element 1 is shown rolled up into a compact size roll which can fit readily into large or small basic first aid kits or similar. When rolled the struts flatten out but once the element is opened for use, they resume the shape, length and strength of an extremely stiff/rigid strut capable of supporting a multitude of limb injuries. Preferably, the splint element 1 will unroll spontaneously under elastic deformation once released to adopt its use configuration.

Element 1 comprises a stiff strut member 2 to which is attached a plurality of flexible straps 3, 4, 5 and 6. Each strap is fixed to stiff strut 2 which comprises a material which is stiff enough to provide splitting but flexible enough to allows folding or rolling up. Depending upon the size of the Element 1 the rolled element could be sized no bigger than the size of a standard bandage. Each splint element can be adapted for single use and discarded or it can be reused as required. When used with open injuries it can be discarded after use for infection control. For closed injuries the splint element can be reused.

Since each element can be rolled up or folded into a compact size, the materials selected for the struts and straps accommodate this objective. Material selected include metals timber, plastics or any other material with sufficient stiffness to act as a splint but with sufficient flexibility for folding or rolling up. Alternative embodiments of the present invention will be described below:

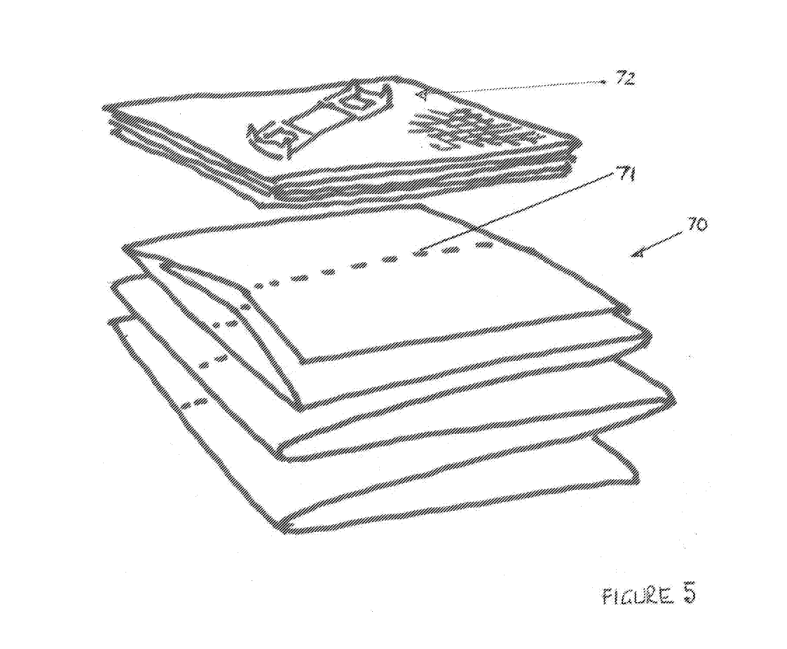

Referring to FIG. 5 shows a folded view of a limb splint assembly 70 according to an alternative embodiment. Splint 70 is shown folded transversely about a longitudinal extent indicated by dotted line 71 and thence folded longitudinally along the line 71. Splint 79 is shown in kit form and includes fasteners 72

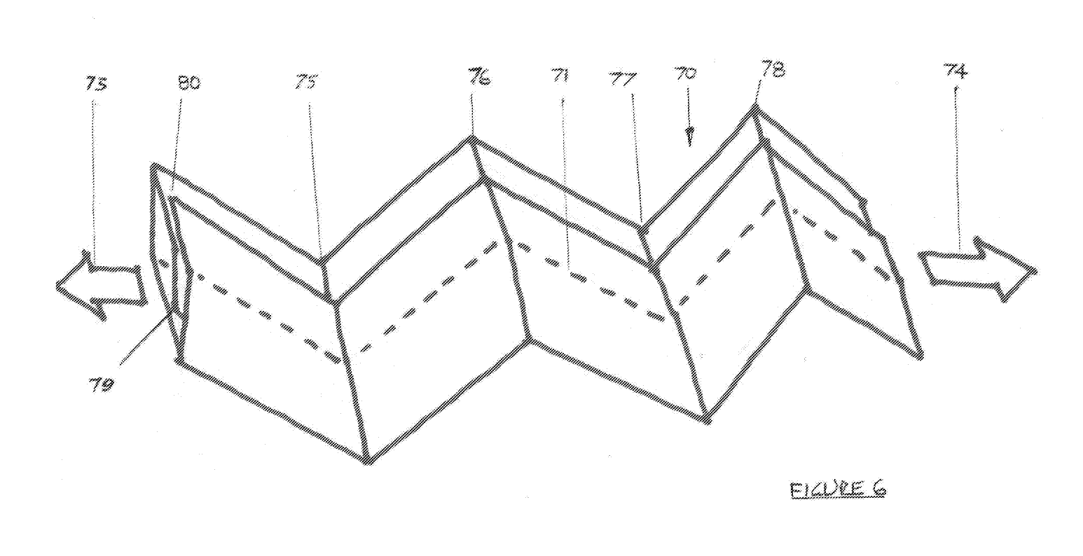

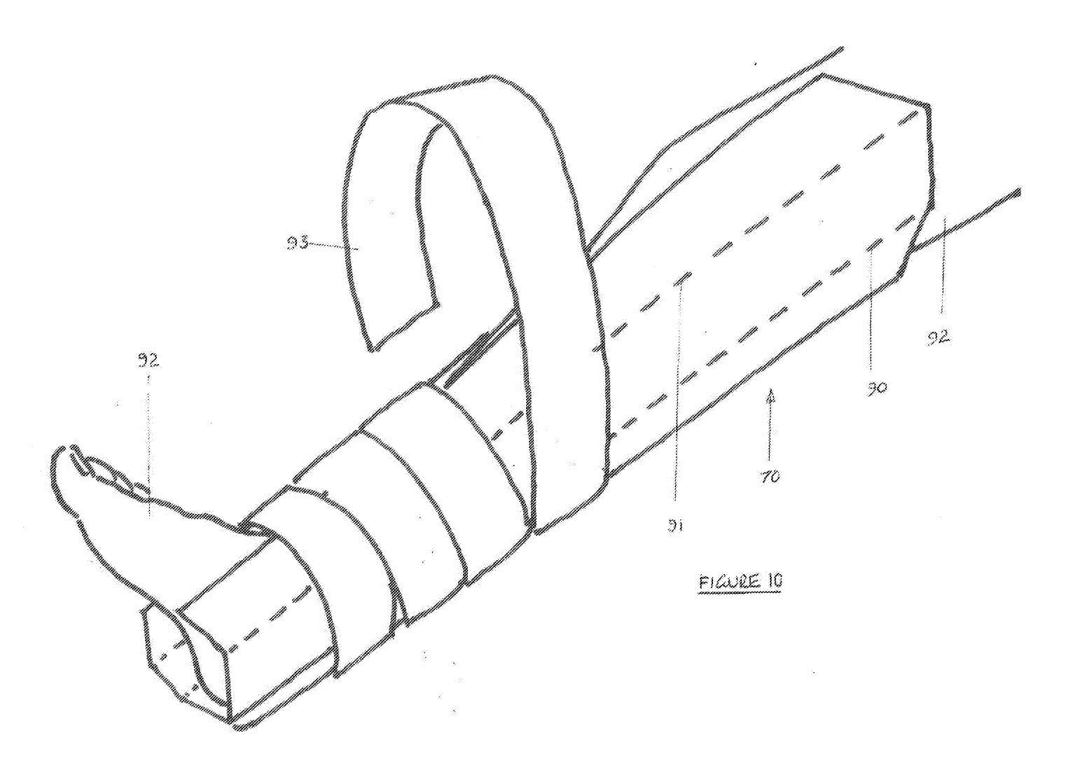

FIG. 6 shows with corresponding numbering the limb splint 70 of FIG. 5 partially unfolded along its longitudinal extent in the direction of arrows 73 and 74, it will be appreciated that the size of the splint can be varied to accommodate different limb sizes. For example a child's splint will be smaller and shorter than one used for a larger adult. Splint 70 has in the example shown folded along transverse fold lines 75, 76, 77, and 78. When in fully developed form the splint 70 is a planar sheet of material suitable for the purpose and which can durably resist continual folding and unfolding (cold working), and be stiff enough to stabilise a limb. Opposing edges 79 and 80 are folded towards each other transversely relative to axis 71 and as required overlap to form an envelope which will accommodate a limb (See FIG. 10).

In preference, the length of the longitudinal plane is approximately half the length of the traverse plane. By way of example, if the length of the traverse plane is 42 centimetres and the length of the longitudinal plane would be 22 centimetres, the first traverse fold would be located approximately 2 centimetres from the first traverse edge and a second traverse fold would be located approximately 10 centimetres from the first traverse edge. The first traverse fold would be located approximately 20 centimetres from the second traverse edge and the second traverse fold would be located approximately 12 centimetres from the second traverse edge. The first and second longitudinal folds and are preferably located an equal distance from each other and the first and second longitudinal edges. For example using the above measurements, the first longitudinal fold would be located approximately 14 centimetres from the first longitudinal edge 28 centimetres from the second longitudinal edge. The second longitudinal fold would be located 28 centimetres from the first longitudinal edge and 14 centimetres from the second longitudinal edge.

It is to be understood that the above measurements are by way of example only and any alternate measurements and ratios could be used depending on the side of the splint 70. Therefore, the splint in the unfolded state can be transformed into the folded state by folding the first traverse edge inward towards the second traverse edge along the first traverse fold, then folded again towards the second traverse edge along the second traverse fold. The fold is on the first longitudinal fold line 71 towards the second longitudinal edge, then a final fold along the second longitudinal fold line 71a inward towards the first longitudinal fold. The splint is then in the folded state as per FIG. 6. In preference, all of the folds are scored or otherwise pre-folded to allow for ease of folding by the user.

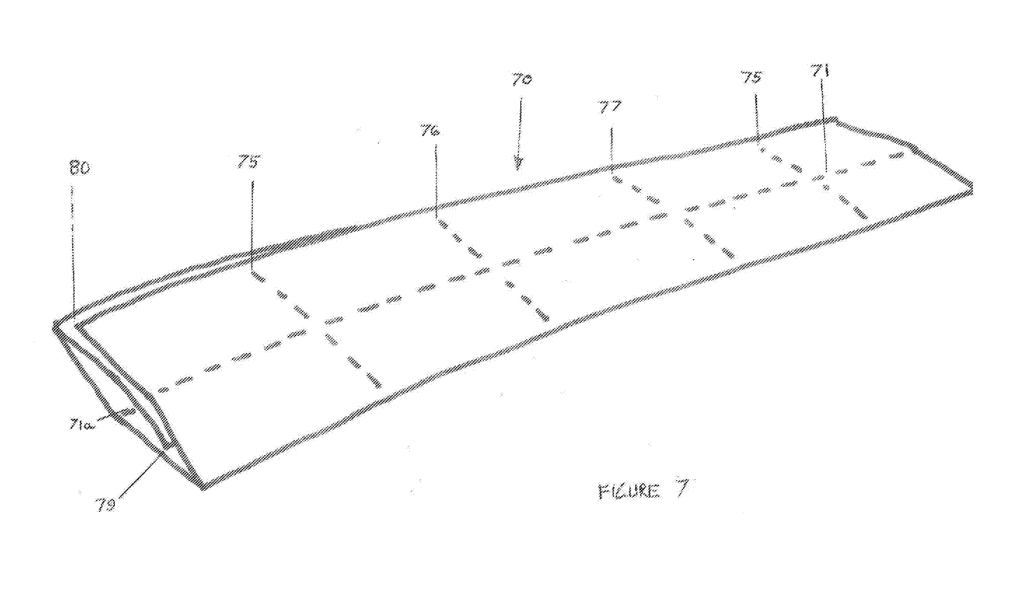

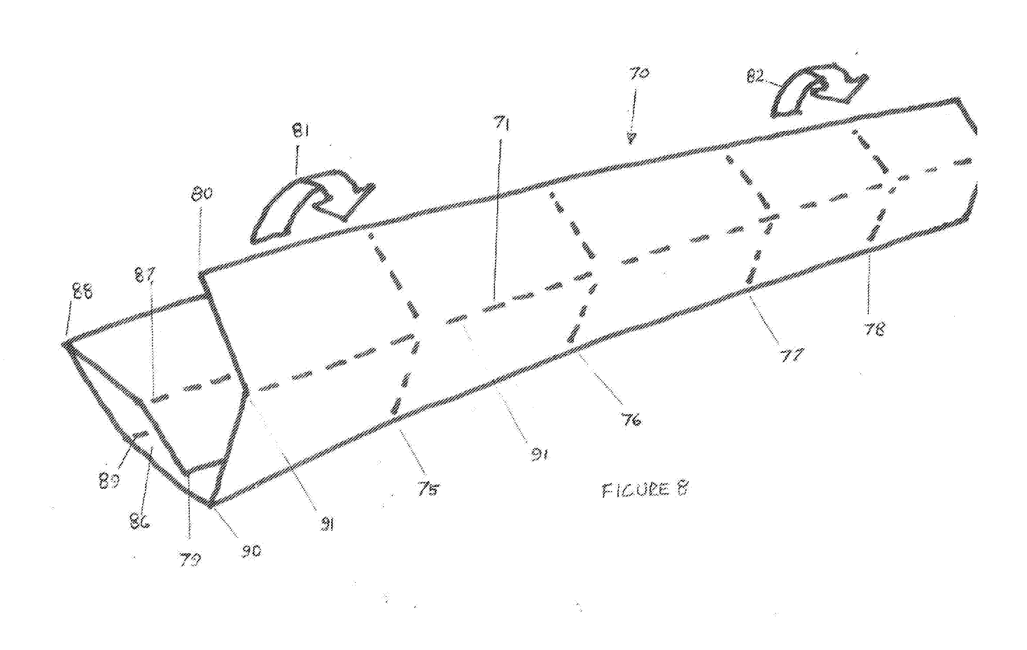

FIG. 7 shows with corresponding numbering, the limb splint of FIG. 5 unfolded along its longitudinal extent. FIG. 8 shows the limb splint 70 of FIG. 7 partially unfolded across its transverse extent in the direction of arrows 81 and 82. Also shown are longitudinal fold lines 87, 88, 89, 90 and 91 about which splint 70 is folded to define internal space 86.

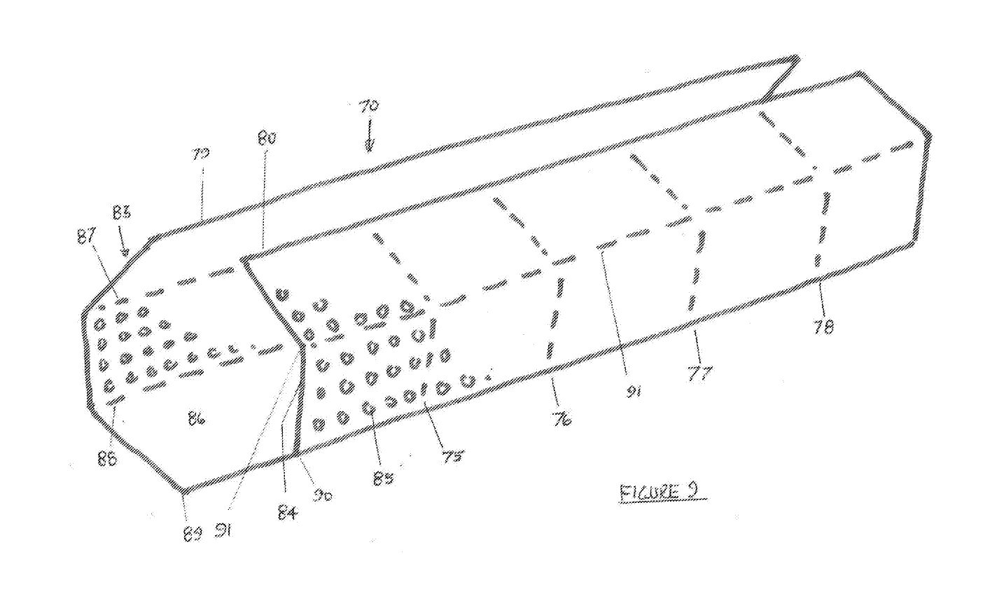

FIG. 9 shows the limb splint 70 of FIG. 7 showing walls 83 and 84 partially unfolded in the transverse direction about a longitudinal axis defining a space 86 in which a limb is placed. Splint 70 includes ‘breathing’ holes 85 which assist circulation of air about the limb. FIG. 9 shows transverse fold lines 75, 76, 77 and 78.

FIG. 10 shows with corresponding numbering the limb splint 70 enveloping a patient leg 92 and secured with a fastening strap 93. According to the embodiment shown strap is wound around the outside of splint 70 in a helical fashion.

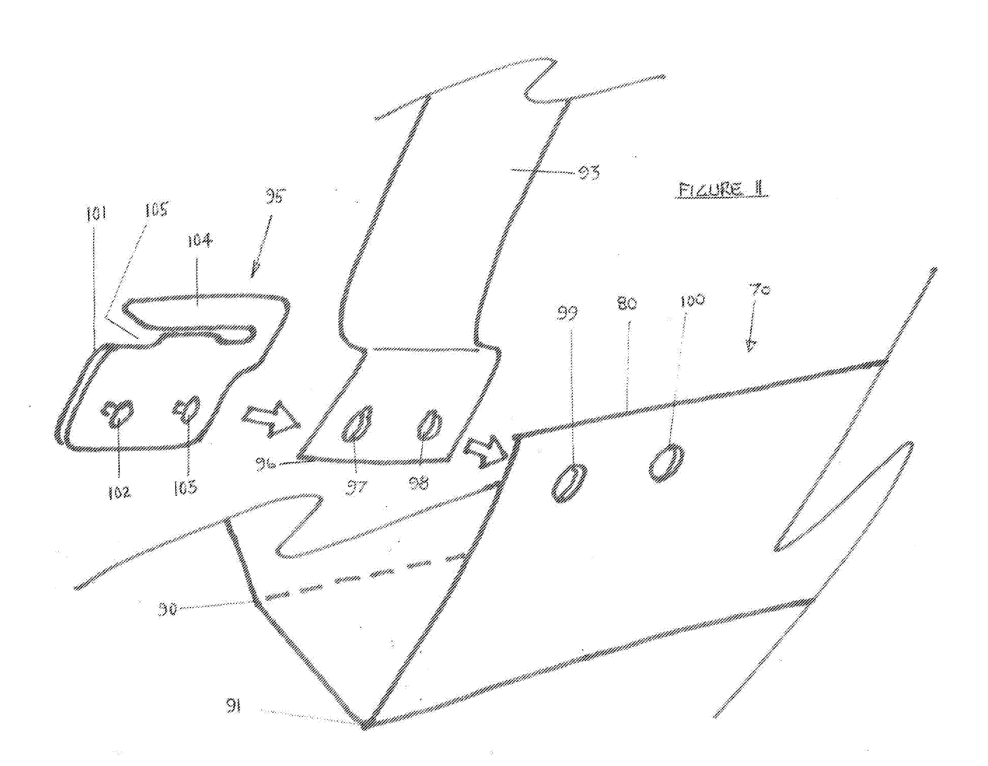

FIG. 11 shows an exploded abbreviated view of the splint 70 with fold lines 90 and 91 and enlarged detail of a connection regime 95 for connecting fastening strap 93 to the splint 70. Strap 93 terminates in end 96 which includes openings 97 and 98 which oppose openings 99 and 100 in splint 70. Retaining clamp 101 includes tabs 102 and 103 which penetrate respective openings 98 and 100 and 97 and 99 and sandwich end 96 of fastening strap 93 between clamp 101 and splint 70. Clamp 101 includes a hook formation 104 which defines a slot recess 105 which receives an opposite end of strap 93 and retains winding tension about splint 70.

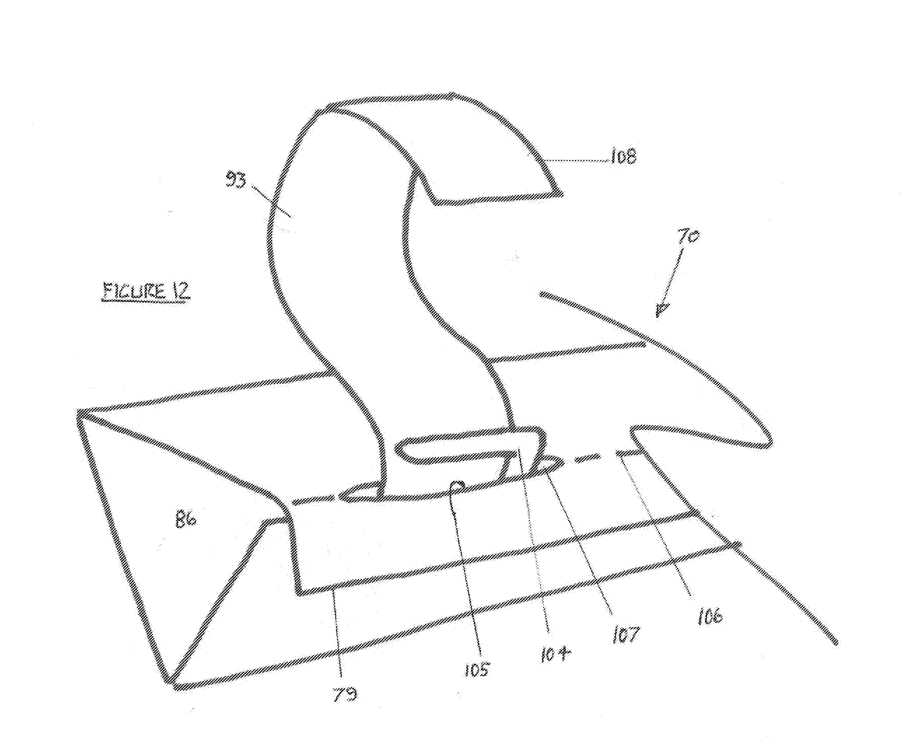

FIG. 12 shows with corresponding numbering an enlarged detail of the connection assembly 95 of FIG. 11 fully engaged. Edge 79 of splint 70 is folded about fold line 106 and includes a slot 107 which receives fastening strap 93. Strap 93 is secured by clamp 101 via hook formation 104. Strap 93 engages recess 105 and is retained by the resilience of hook formation 104. As hook formation is attached to splint 70, this provides anchorage for strap 93 when it is fed up through slot 107 and fastened via hook formation 104.

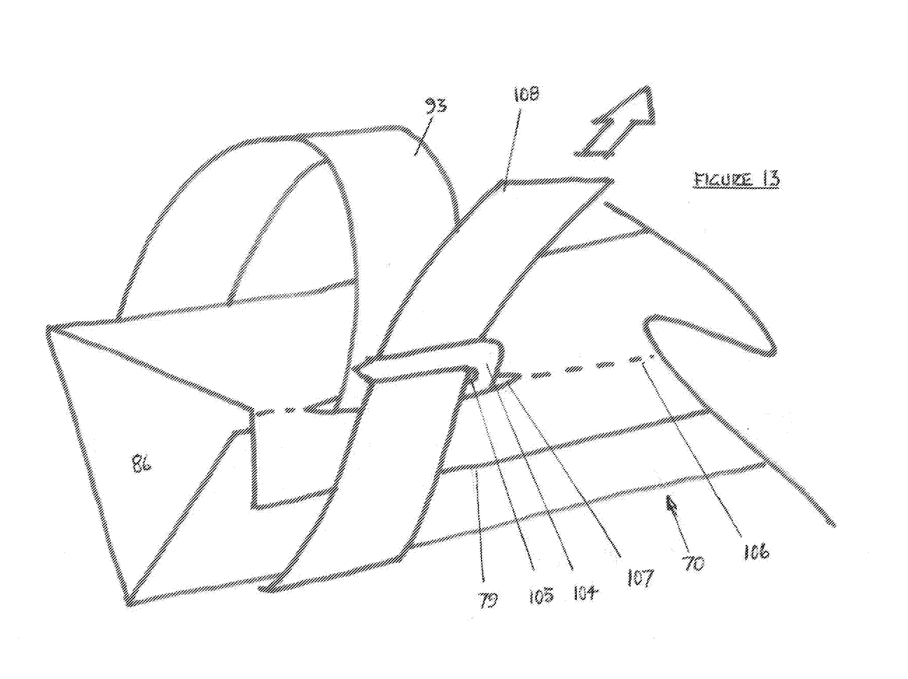

FIG. 13 shows with corresponding numbering an abbreviated enlarged view of the splint 70 and fastening strap 93 extending through slot 105 and wound around the outside of splint 70.

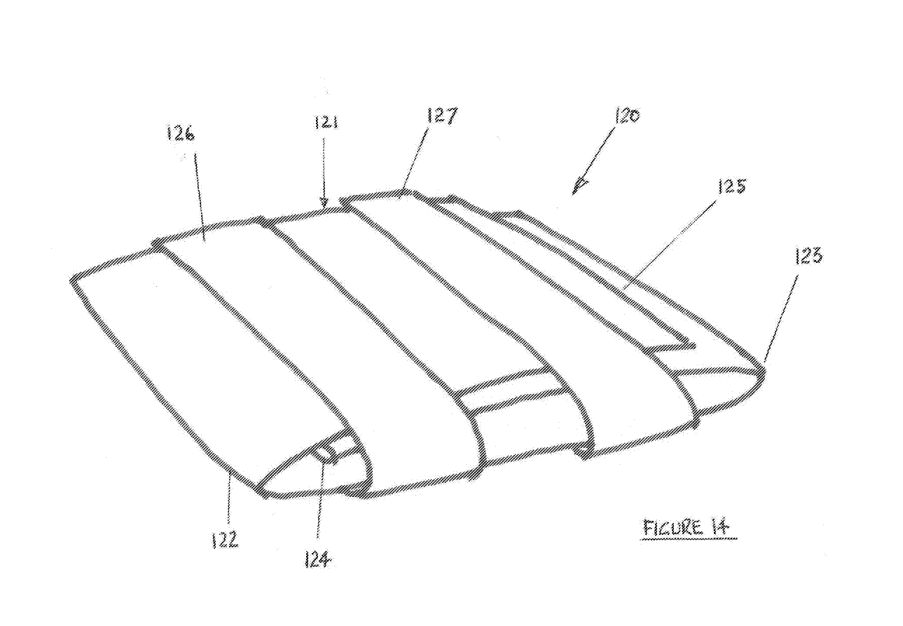

FIG. 14 shows a fully folded limb splint assembly 120 according to a further alternative embodiment. Splint 121 is folded longitudinally about fold lines 122 and 123 such that ends 124 and 125 overlap. Splint 121 is secured in its folded position by straps 126 and 127 which are anchored to splint 121 in a similar manner to that which was described with reference to splint 70. This folding provides a very compact stowable splint assembly for use in first aid kits, paramedics, ambulance kits and the like.

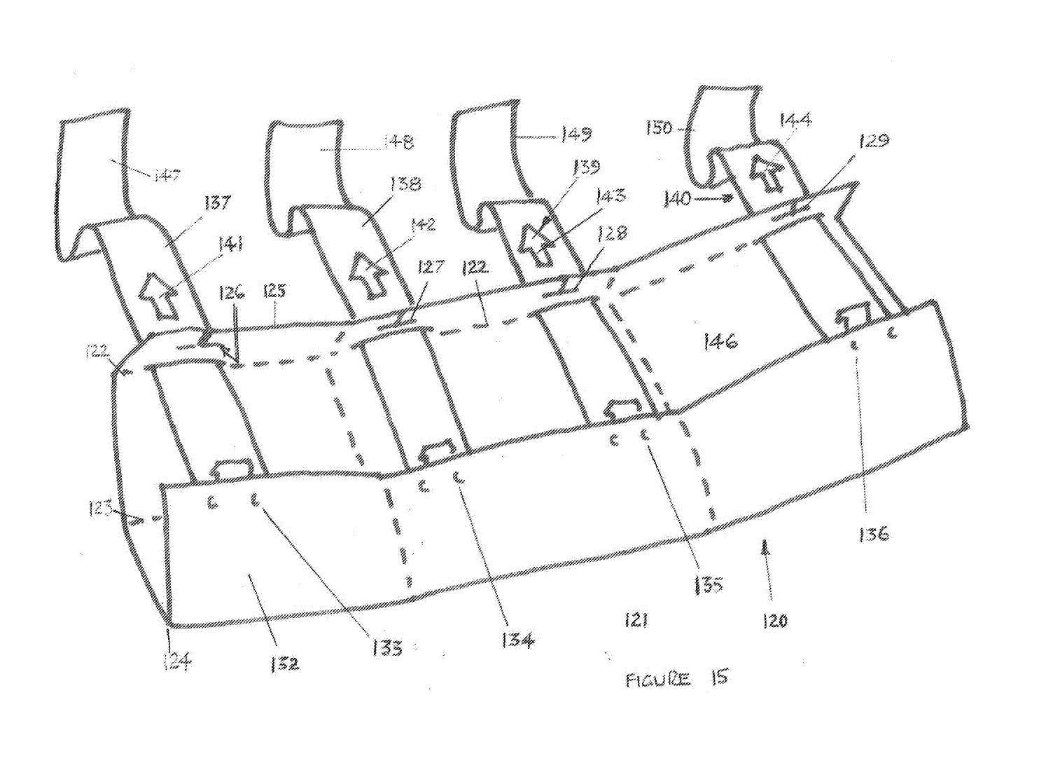

FIG. 15 shows the limb splint assembly 120 of FIG. 14 partially unfurled and including fastening straps. Splint 121 is shown unfolded about longitudinal fold lines 122, 123, 124. Edge 125 is folded about fold line 122 in which there are disposed a plurality of slots 126, 127, 128 and 129. Splint 121 is unfolded longitudinally to its fullest extent about fold lines 130 and 131. Wall 132 receives and retains anchors 133, 134, 135 and 136 which anchor respective fastening straps 137, 138, 139 and 140. Straps 137, 138, 139 and 140 pass through respective slots 126, 127, 128 and 129, in the direction of arrows 141, 142, 143 and 144.

When a limb is placed in internal space 146, respective ends 147, 148, 149 and 150 of fastening straps 137, 138, 139 and 140 may be pulled to tighten splint 121 about a limb and finally engage respective anchors 133, 134, 135 and 136. The embodiment of FIG. 15 contemplates that the splint assembly 120 receives a limb in space 146 so that splint 121 envelops die limb ( not shown). Thus is this embodiment, the limb will be underneath fastening straps 137, 138, 139 and 140. In this case ends 147, 148, 149 and 150 could be passed under the splint 121 then each back on themselves for fastening via anchors 133, 134, 135 and 136.

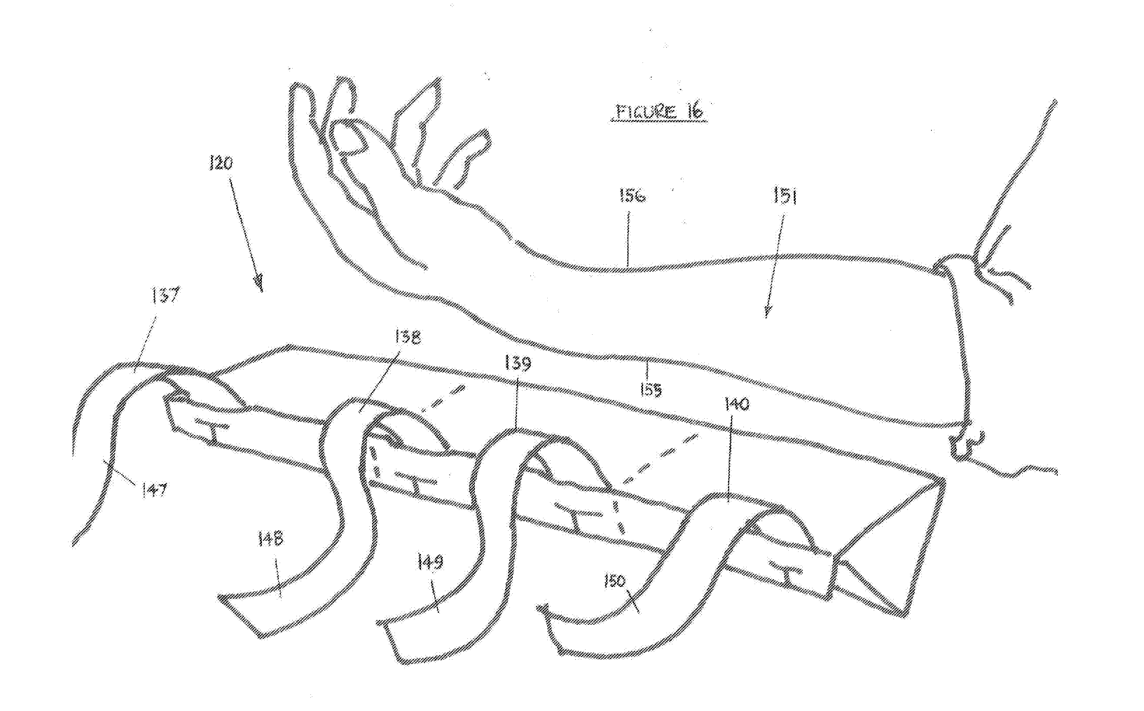

FIG. 16 shows with corresponding numbering, the splint assembly 120 of FIG. 15 presented to an arm 151 of a patient prior to engagement. In this embodiment the arm may be restrained in an alternative manner of fixation. In this case the splint is laid along the back 155 of the arm with the fastening straps 137, 138, 139 and 140 wound over the front of the arm. In this case the splint could be folded flat and placed against and along the back 155 of the arm 151. Fastening straps 137, 138, 139 and 140 would then be wound around the arm and back on themselves at a selected tension. This may be achieved by co-operation between ends 147, 148, 149 and 150 of fastening straps 137, 138, 139 and 140.

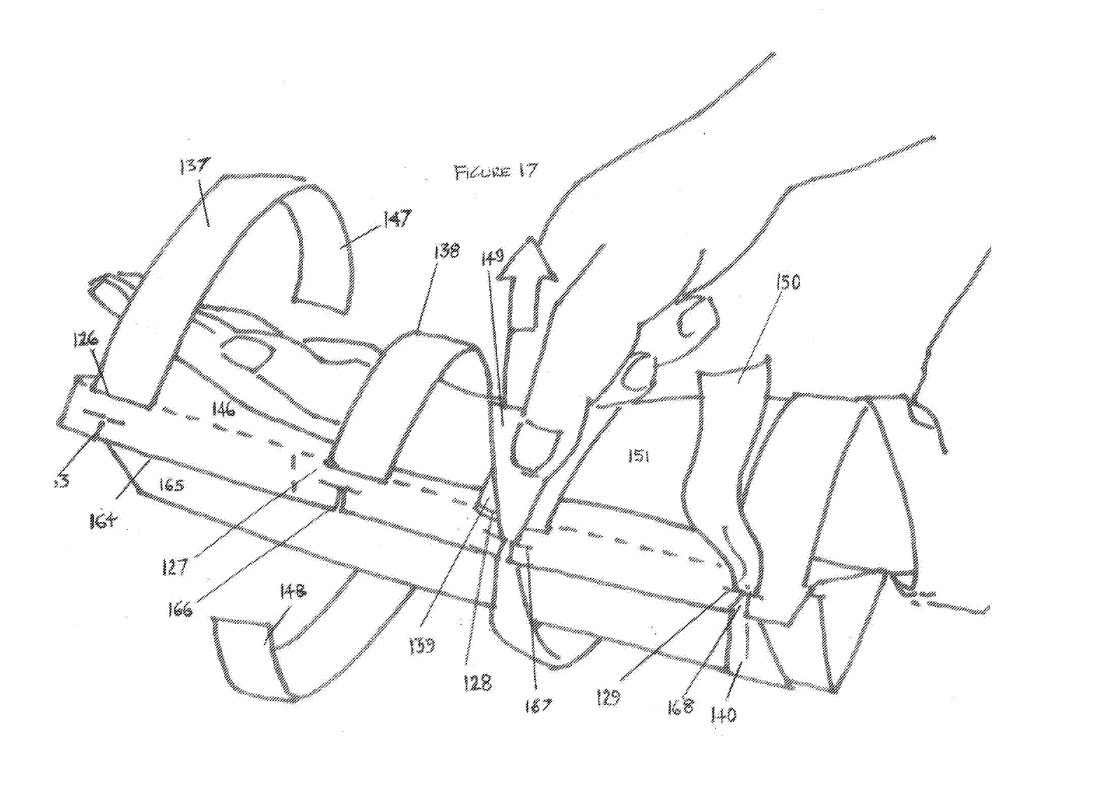

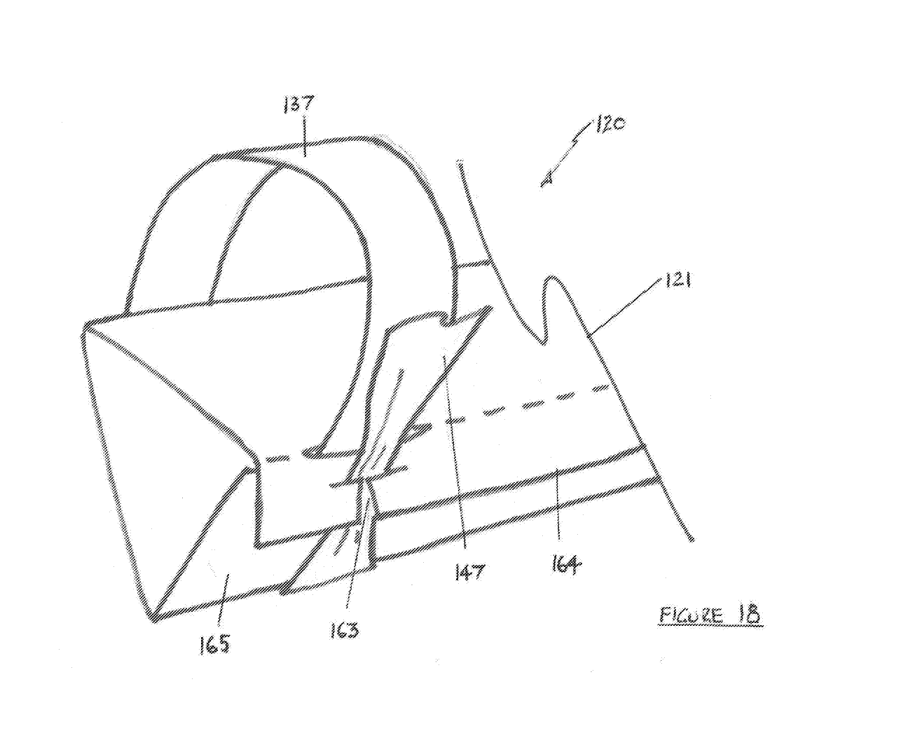

FIG. 17 shows with corresponding numbering the splint assembly of FIG. 15 partially installed on a patient arm 151. In this embodiment, the fastening straps 137, 138, 139 and 140 are wound over the front of the arm 151. Straps 161 is anchored to wall 165 in a similar manner to that which was described previously with reference to FIG. 13. Splint 121 further comprises locking slots 163, 166, 167 and 168 whose operation will be described with reference to FIG. 18 below.

FIG. 18 shows an enlarged abbreviated view of the splint assembly 120 and the engagement between a fastening strap 137. In this embodiment, end 147 which has now been fully wound around splint 121 of strap 137 engages locking slot 163 cut into edge 164. This allows quick fixation and release and also allows tensioning while the end 147 of strap 137 remains in slot 163. Slot 163 has sufficient flexibility to allows quick engagement and release of fastener 137 and applies sufficient friction to retain tension when the splint 121 is fitted to a limb. In this embodiment, strap 137 is anchored to wall 165 in a similar manner to that which was described previously with reference to FIG. 13. End 147 can be pulled through slot 163 with increasing tension applied about a limb to secure the splint which as much tension as is required.

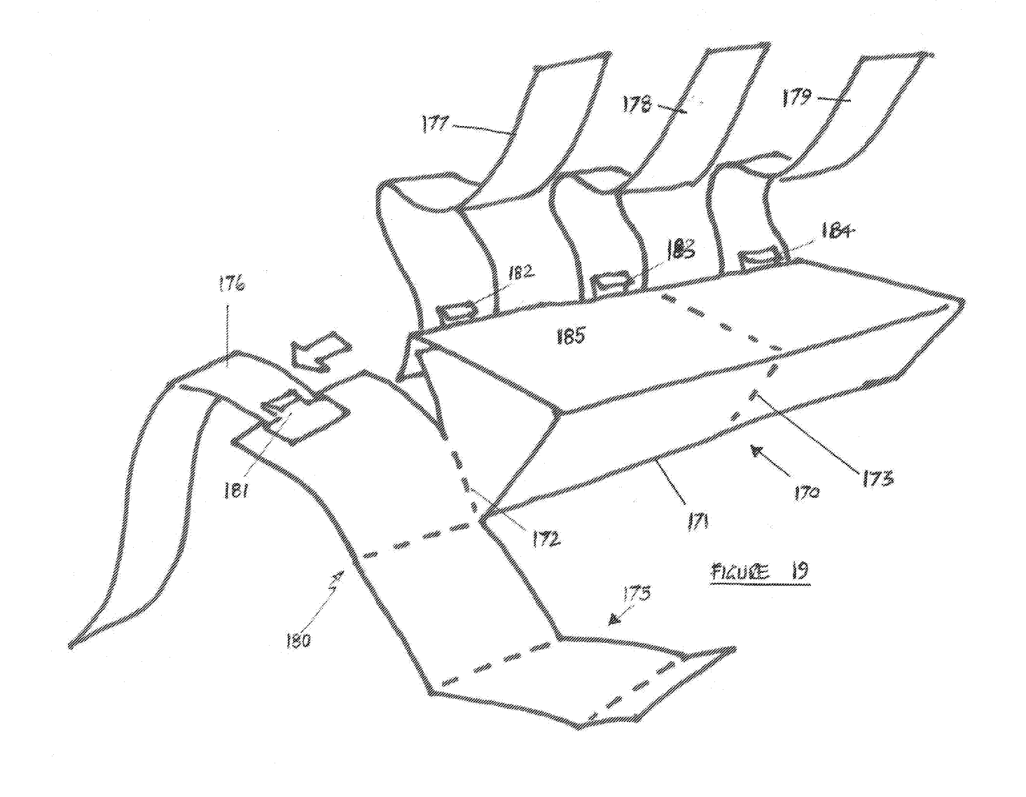

FIG. 19 shows a perspective view of a segmented limb splint assembly 170 according to a further alternative embodiment. In this embodiment, it can be seen that splint 171 can be cut along transverse fold lines 172 and 173 to form either a series of smaller splints or one which better conforms to the shape and/or size of a limb. Splint 171 is shown cut along fold line 172 potentially enabling it to be disposed at a different angle relative to a longitudinal axis. Wall section 175 is opened out and the size of an internal space adjusted as required. As before, once the configuration of splint 171 is decided by a user, the splint can be secured to a limb using fasteners 176, 177, 178 and 179 which can be looped around splint 171 and reconnected. Fasteners 176, 177, 178 and 179 are respectively secured by clamps 181, 182, 183 and 184 attached to wall 185 in a similar manner to that described earlier. Preferably a plastics material is used for the splint material. The strength of the plastic and the tension created by the interconnection of the fasteners which engage the slots, secures the splint in the “in use” state. In this state the splint 171 comprises a substantially triangular shaped recess.

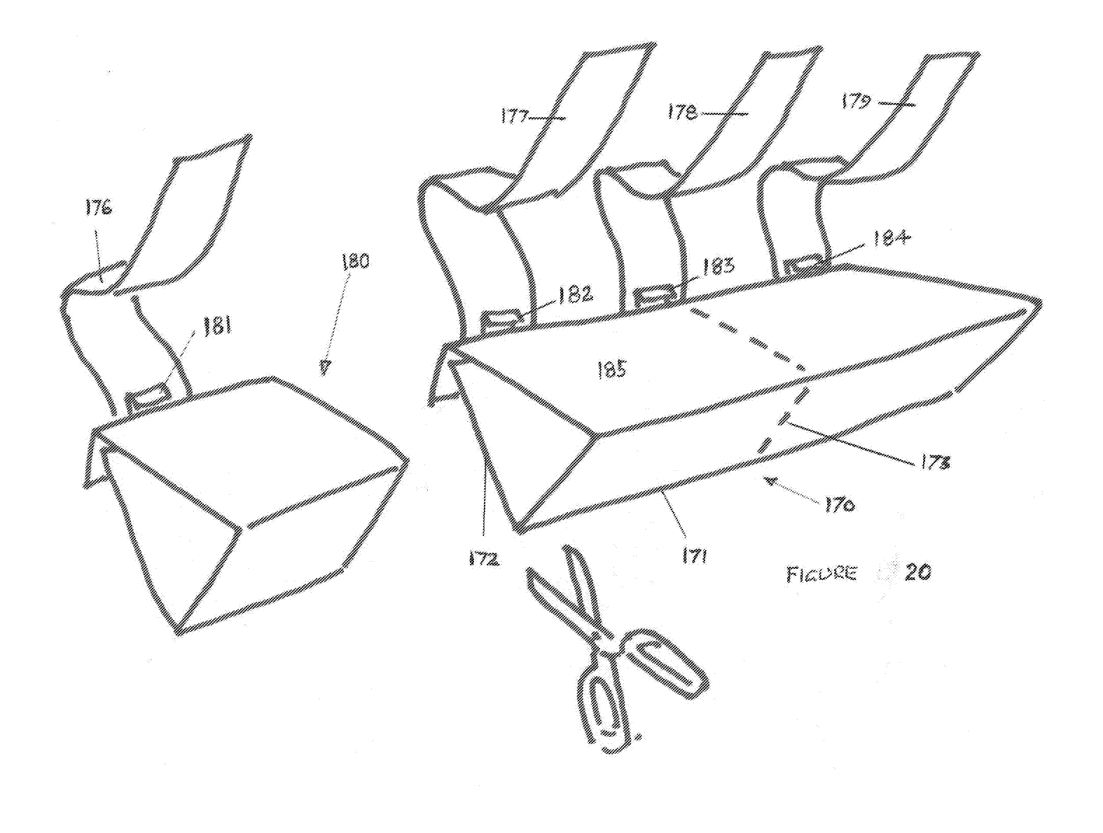

FIG. 20 shows with corresponding numbering, the limb splint 171 of FIG. 19 with a segment 180 isolated (by scissor cut) from the remainder of splint 171.

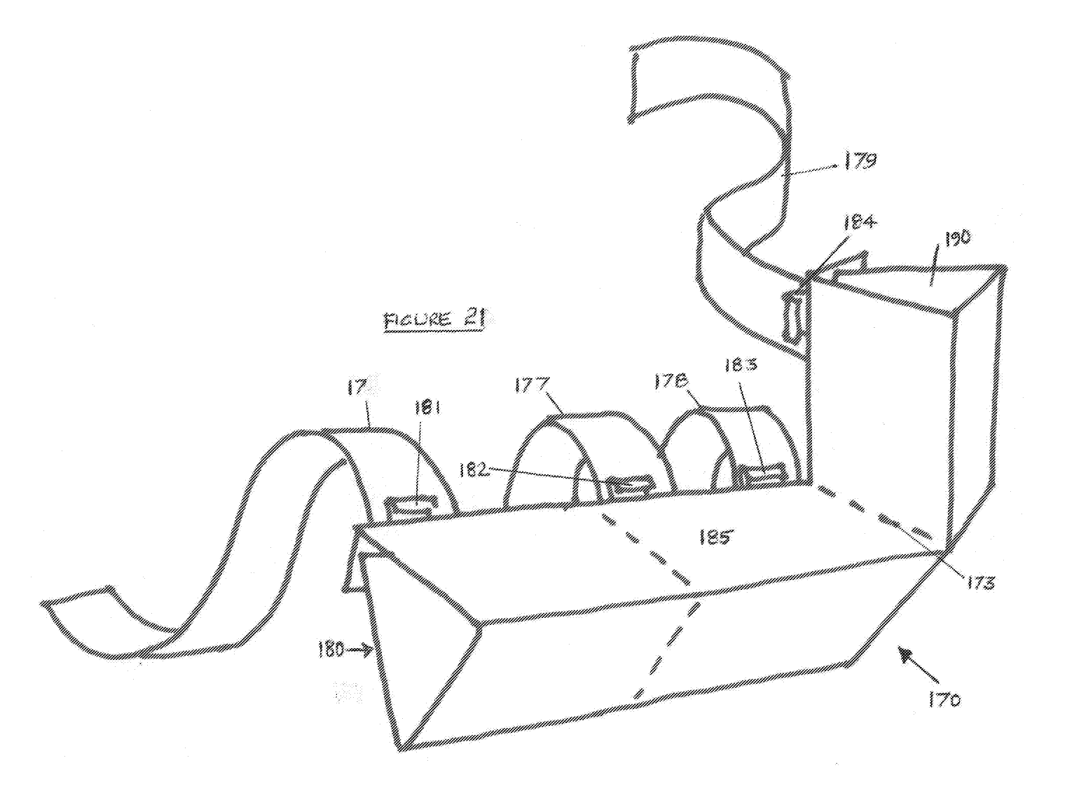

FIG. 21 shows the segmented limb splint 171 in a configuration suitable for use in spinning an arm. In this case, segment 190 would be suitable to wrap about a Humerus so that a patient can cock the elbow. This might be useful where a patient needs to wear a sling. With the fold lines and the flexibility of the splint material various configurations may be adopted depending upon particular requirements. Additional segments can be introduced for long arm and segments reduce for short arms—such as those of a child. The limb can be laid on the outside of the splint or within a recess created by walls of the splint to both cases the fasteners apply the required tension.

The present invention as described herein retains the advantages of a conventional splint but improves patient comfort during application and allows rescuers to apply the splint with safety and without discomfort to the patient. It also, allows earlier management of limb fractures reducing pain and reducing treatment time increasing patient security from further damage. The splint is simple to operate, can be attached with minimal pain, is safer and adjustable to accommodate patients of different sizes. The splint is fitted with straps to secure the legs of a patient. This, allows the patient's limb to be fully restrained from movement and allows rescue services to evacuate the patient quickly.

It will be appreciated by persons skilled in the art that although the example described with reference to the illustrations show a assemblies which include a limb splint, it will be appreciated that the assembly and disassembly technique described can be adapted for alternative limb splints.

Further advantages and improvements may very well be made to the present invention without deviating from its scope. Although the invention has been shown and described in what is conceived to be the most practical and preferred embodiment, it is recognized that departures may be made therefrom within the scope and spirit of the invention, which is not to be limited to the details disclosed herein but is to be accorded the full scope of the claims so as to embrace any and all equivalent devices and apparatus. Any discussion of the prior art throughout the specification should in no way be considered as an admission that such prior art is widely known or forms part of the common general knowledge in this field.

Persons skilled in the art will now appreciate the present invention which provides for a foldable, reusable splint which can be applied easily by a lay person or medical professional to assist in the treatment of injuries. The stiff material of construction provides a strong, secure splint that will provide support to the victim and keep their injury immobilised. The invention improves upon known splints because it is easy to use; it can be folded into smaller dimensions for convenient storage and transport; it can be used on various parts of the body; and it is reusable.

The splints described herein can also be used on different parts of a human or animal body. The invention is therefore adaptable and an improvement over known splints which are directed for use on particular body parts. For example, in the known art a wrist splint can only be used on the wrist and a leg splint can only be used on a leg. According to the present invention the splint can be used on different limbs such as an arm or leg but in an alternative embodiment the compact splint can be especially adapted for use on a particular limb. Preferably, the splint when used for an arm, includes a tuck in fastening for securing the fastening straps. When used for a leg splint the same or similar materials of manufacture are used but employs a fabric bandage for strapping the limb to the splint. Thus the way the foldable splint is used can be adjusted according to the part of the body on which it is used. Alternatives are contemplated for the securing clips. One described employs a clamping hook and another contemplates a slot cut into the splint material to allow convenient engagement and disengagement and pull through for tensioning. One preferred material (but non limiting) of construction for the splint is Polypropylene in the range of about 0.5-1.5 mm thick. Injection moulded plastics such as ABS or similar are suitable for the splint and clamps. The fastening straps are preferably manufactured from Nylon or similar material and are preferably around 0.2-0.4 mm thick. Other thicknesses are contemplated.

In the present specification and claims (if any), the word “comprising” and its derivatives including “comprises” and “comprise” include each of the stated integers but does not exclude the inclusion of one or more further integers.

It will be appreciated by persons skilled in the art that the invention described herein is also adaptable to alternative applications. It will be recognised by persons skilled in the art that numerous variations and modifications may be made to the invention as broadly describe herein without departing from the spirit and scope of the invention. For example the fasteners can be engaged by hook and loop connections (sometimes known as VELCRO™), clips, buckles, ratchets, and the like or any connection creating hoop tension.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the description of embodiments herein.

1. A portable splint assembly for fixation to a limb; the splint assembly comprising at least one elongated member having first and second free ends; and at least one fastener which enables fastening of the splint to a limb; wherein the at least one elongated member has a long axis along its longitudinal extent extending between said free ends and at least one transverse shorter axis; and wherein the elongated member is resiliency deformable to allow folding and/or or rolling about at least one said axes.

2. A splint assembly according to claim 2 wherein the at least one elongated member moves between a first state in which the elongated member is completely folded or rolled up and a second state in which the at least one elongated member is unfolded or unrolled to enable working engagement with a limb.

3. A splint assembly according to claim 2 wherein the at least one elongated member is foldable about long and shorter transverse axes

4. A splint assembly according to claim 3 wherein the at least one elongated member has multiple fold lines disposed transversely to a longitudinal axis.

5. A splint assembly according to claim 4 wherein the at least one elongated member has at least one longitudinal fold line about which the elongated member is folded.

6. A splint assembly according to claim 5 wherein the elongated member comprises a plurality of struts.

7. A splint assembly according to claim 6 wherein the plurality of struts are connected by fastening straps which secure the struts against a limb.

8. A splint assembly according to claim 7 wherein opposing ends of each fastening strap overlap and engage by a releaseable connection.

9. A splint assembly according to claim 8 wherein hoop tension is generated about the limb when each fastening strap is secured.

10. A splint assembly according to claim 9 wherein the hoop tension is adjustable by adjusting an extent of overlap of the ends of the fasteners.

11. A splint assembly according to claim 10 wherein the fasteners are formed from a flexible material which is capable of conformity to a variety of limb sizes and shapes.

12. A splint assembly according to claim 11 wherein the assembly when unfolded comprises a plurality of parallel and spaced apart struts connected by a plurality of transversely disposed fastening members.

13. A splint assembly according to claim 12 wherein extremities of each fastener extend beyond the outermost of the parallel struts.

14. A splint assembly according to claim 13 wherein the transversely disposed fasteners are spaced apart along the longitudinal extent of the struts.

15. A splint assembly according to claim 14 wherein the fasteners engage via hook and loop connection.

16. A splint assembly according to claim 15 wherein the elongated member is capable of being rolled up about an axis transverse to a longitudinal axis of the elongated member.

17. A splint assembly according to claim 16 wherein fasteners are tied around the rolled up elongated member to secure the elongated member in its rolled up form.

18. A splint assembly according to claim 17 wherein the elongated member is foldable multiple times about at least one long fold line and multiple times about transverse fold lines.

19. A splint assembly according to claim 5 wherein the elongated member is formed from a planar sheet of flexible material which is resiliently deformable.

20. A splint assembly according to claim 19 wherein the planar sheet includes a plurality of fold lines, at least one of which runs longitudinally along a long dimension and at east one of which runs transverse across a shorter dimension.

21. A splint assembly according to claim 20 wherein the planar sheet when partially unfolded defines a space which forms a saddle and receives and retains a limb.

22. A splint assembly according to claim 21 wherein the space is formed by folding opposing edges of the planar sheet inwardly toward each other about one or more of the long fold lines.

23. A splint assembly according to claim 22 wherein the elongated member includes associated therewith at least one fastening strap.

24. A splint assembly according to claim 23 wherein when the assembly is fully folded, its outside dimensions are at least 50% less than outside dimensions of the elongated member when fully unfolded.

25. A splint assembly according to claim 24 wherein when folded the planar member is secured and retained in the folded state by at least one of said fasteners.

26. A splint assembly according to claim 25 wherein the fasteners wrap about the outside of the folded planar member when the assembly is fully folded.

27. A splint assembly according to claim 26 wherein the elongated member includes a plurality of slots which receive and releasably retain cooperating tabs which align with the slots.

28. A splint assembly according to claim 27 wherein when the tabs engage the slots, the splint defines an internal space which accommodates a limb of a patient.

29. A splint assembly according to claim 28 wherein, the splint includes at least one retaining clamp which each retain a cooperating fastening strap.

30. A splint assembly according to claim 29 wherein, each said retaining clamp is securely attached to a wall of the splint and includes a hook which receives and retains an end of the fastener.

31. A splint assembly according to claim 30 wherein, each fastening strap terminates in an end which includes openings which oppose corresponding openings in the splint to facilitate anchorage to a wall of the splint.

32. A splint assembly according to claim 31 wherein, the retaining clamp includes tabs which penetrate respective openings in the fastener and a wall of the splint to effect said anchorage.

33. A splint assembly according to claim 32 wherein, the retaining clamps each includes hook formations which retain the fasteners to secure a limb.

34. A splint assembly according to claim 33 wherein the hook formations each receive an opposite end of one said straps to retain winding tension about the splint.

35. A splint assembly according to claim 34 wherein, the internal space defines one of a channel, half cylindrical dome, circle, rectangle, U shape.

36. A splint assembly according to claim 35 wherein the splint is manufactured from a plastics material.

37. A splint assembly according to claim 32 wherein, the splint includes edge cuts which enable releasable engagement and disengagement of the fastening straps.
